(19) United States
(12) Patent Application Publication
(10) Pub. No.: US 2011/0208071 A1
(43) Pub. Date: Aug. 25, 2011

(54) SMART NON-INVASIVE ARRAY-BASED HEMODYNAMIC MONITORING SYSTEM on CHIP AND METHOD THEREOF

(75) Inventors: Liang-Hung Lu, Taipei (TW); Wei-Cheng Tian, Taipei (TW); Shao-Yi Chien, Taipei (TW); Jiun-Lang Huang, Taipei (TW); Po-Ling Kuo, Taipei (TW); Ping-Cheng Yeh, Taipei (TW)

(73) Assignee: National Taiwan University, Taipei (TW)

(21) Appl. No.: 13/034,011

(22) Filed: Feb. 24, 2011

Related U.S. Application Data
(60) Provisional application No. 61/307,488, filed on Feb. 24, 2010.

(51) Int. Cl. A61B 5/02

(52) U.S. Cl. 600/500

(57) ABSTRACT

A non-invasive array-based hemodynamic monitoring system on chip is disclosed. The non-invasive array-based hemodynamic monitoring system on chip comprises a CMOS MEMS pressure sensor array, a readout circuit, and a signal control system. The CMOS MEMS pressure sensor array is configured to sense a pulse wave of a blood vessel. The readout circuit is coupled with each of the CMOS compatible MEMS pressure sensors and is configured to read the pulse wave and transformed the pulse wave into a voltage signal. The signal control system is coupled with each of the readout circuit, and is configured to estimate a wave velocity according to the voltage signal.

### CROSS-REFERENCE TO RELATED APPLICATIONS

This application claims the benefit of U.S. Provisional Application No. 61/307,488, filed on Feb. 24, 2010 and entitled SMART NON-INVASIVE ARRAY-BASED HEMODYNAMIC MONITORING SYSTEM ON CHIP (SOC), the entire contents of which are incorporated herein by reference.

### BACKGROUND OF THE INVENTION

1. Field of the Invention

The present invention generally relates to hemodynamic monitoring system, and more particularly to a smart non-invasive array-based hemodynamic monitoring system-on-a-chip (SoC) and method thereof.

2. Description of Related Art

Nowadays, a cerebrovascular accident (CVA) which is also referred to as a stroke is considered one of the major threats to our health. As the disturbance in the blood supply to the brain caused by a blocked blood vessel, the affected area of the brain is unable to function, leading to inability of body movement or speech. Conventionally, the blood flow in carotid arteries is typically adopted as an indicator to evaluate the risk of a cerebrovascular accident. Based on current technologies, the most widely used tool for this measurement is the doppler ultrasound flow meter. However, the instrument is very expensive and requires experienced technician to operate. The measurement is only limited to hospitals or medical centers.

Due to the disadvantage of conventional hemodynamic monitoring device or technique, a need has arisen to propose a novel hemodynamic monitoring device, system and method for non-invasive carotid blood flow test based on the SoC approach.

### SUMMARY OF THE INVENTION

In view of the foregoing, it is an object of the embodiment of the present invention to provide a smart non-invasive array-based hemodynamic monitoring system-on-a-chip (SoC) and method thereof to estimate the blood rate and healthy condition easily.

According to one embodiment, a non-invasive array-based hemodynamic monitoring system on chip is disclosed. The non-invasive array-based hemodynamic monitoring system on chip comprises a complementary metal-oxide-semiconductor microelectromechanical systems (CMOS MEMS) pressure sensor array, a readout circuit, and a signal control system. The CMOS MEMS pressure sensor array is configured to sense a pulse wave of a blood vessel. The readout circuit is coupled with each of the CMOS compatible MEMS pressure sensors and is configured to read the pulse wave and transformed the pulse wave into a voltage signal. The signal control system is coupled with each of the readout circuit, and is configured to estimate a wave velocity according to the voltage signal.

According to another embodiment, a non-invasive array-based hemodynamic monitoring method is disclosed. The method includes the following steps: firstly, a CMOS MEMS pressure sensor array is provided to sense a pulse wave of a blood vessel. Then, the pulse wave is transformed into a voltage signal. Finally, a waveform estimation algorithm is executed to estimate a wave velocity according to the voltage signal.

According to further embodiment, a CMOS compatible MEMS pressure sensor is disclosed. The CMOS compatible MEMS pressure sensor comprises a top flexible plate, a bottom electrode plate, and a cushion electrode plate. The cushion electrode plate is coupled with the top flexible plate and keeping flat between the top flexible plate and the bottom electrode plate, wherein when the top flexible plate is pressed, the electrical characteristics (such as capacitance change) among the top flexible plate, the bottom electrode plate and the cushion electrode plate indicates the pressure quantity.

### BRIEF DESCRIPTION OF THE DRAWINGS

FIG. 1 shows a diagram illustrating the application of a non-invasive array-based hemodynamic monitoring device according to one embodiment of the present invention;

FIG. 2 shows a system architecture diagram illustrating a n on-invasive array-based hemodynamic monitoring SoC according to one embodiment of the present invention;

FIG. 3 shows a diagram illustrating waveforms sensed by the sensors on a sensor array according to one embodiment of the present invention;

FIG. 4A shows a diagram illustrating initial multi-bank memory according to one embodiment of the present invention;

FIG. 4B shows a diagram illustrating usage of multi-bank memory according to one embodiment of the present invention;

FIG. 4C shows a diagram illustrating usage of multi-bank memory according to another embodiment of the present invention;

FIG. 5A shows a diagram illustrating design of a capacitive pressure sensor according to one embodiment of the present invention;

FIG. 5B shows a diagram illustrating design of a piezo-resistive pressure sensor according to one embodiment of the present invention; and

FIG. 6 shows a flow diagram illustrating a non-invasive array-based hemodynamic monitoring method according to one embodiment of the present invention.

### DETAILED DESCRIPTION OF THE INVENTION

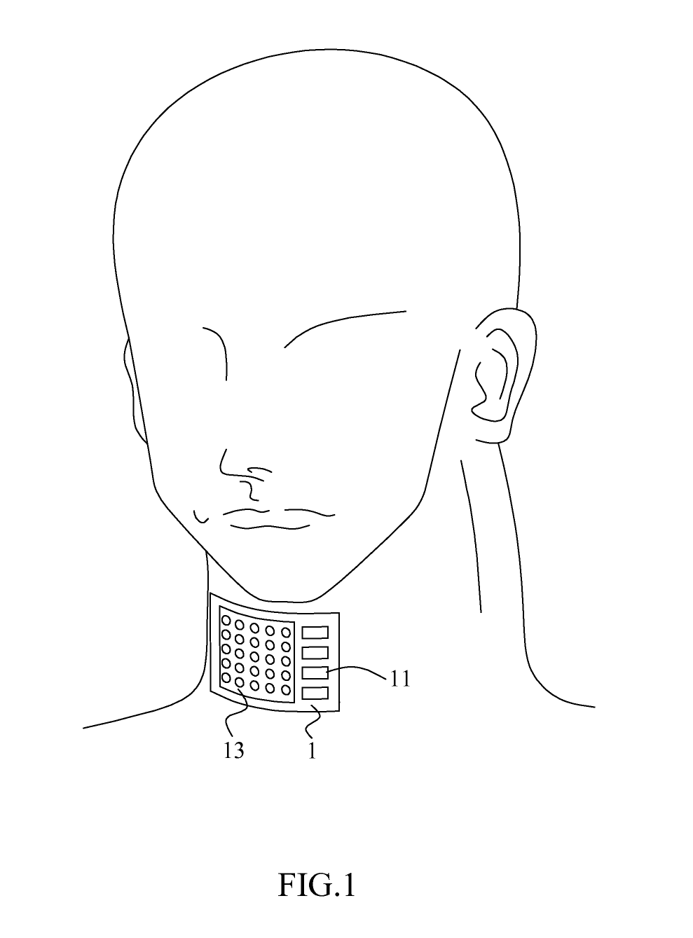

FIG. 1 is a diagram illustrating the application of a non-invasive array-based hemodynamic monitoring device according to one embodiment of the present invention. As shown in FIG. 1, the non-invasive array-based hemodynamic monitoring device 1 may be like a paster which can be stuck on the skin of a patient to predict the wave form and the wave velocity of the pulsatile pressure propagating in a blood vessel, such as an artery, and the rate of blood flow in the artery. It comprises a CMOS MEMS pressure sensor array 13 for sensing the pulse wave and several control units 11 for whole operations such as model or parameter setup.

This proposed medical equipment, non-invasive array-based hemodynamic monitoring device 1, can be automatically calibrated to measure the patient's arterial blood flow rate based on the waveform of the pulse pressure and pulse wave velocity (PWV). The blood flow rate is an important indicator in the treatments of cardiovascular diseases nowadays. The proposed integrated medical equipment can monitor the blood flow of the patients continuously for a long period, and the condition of the patients can be evaluated by analyzing the collected data, as discussed in more detail below.

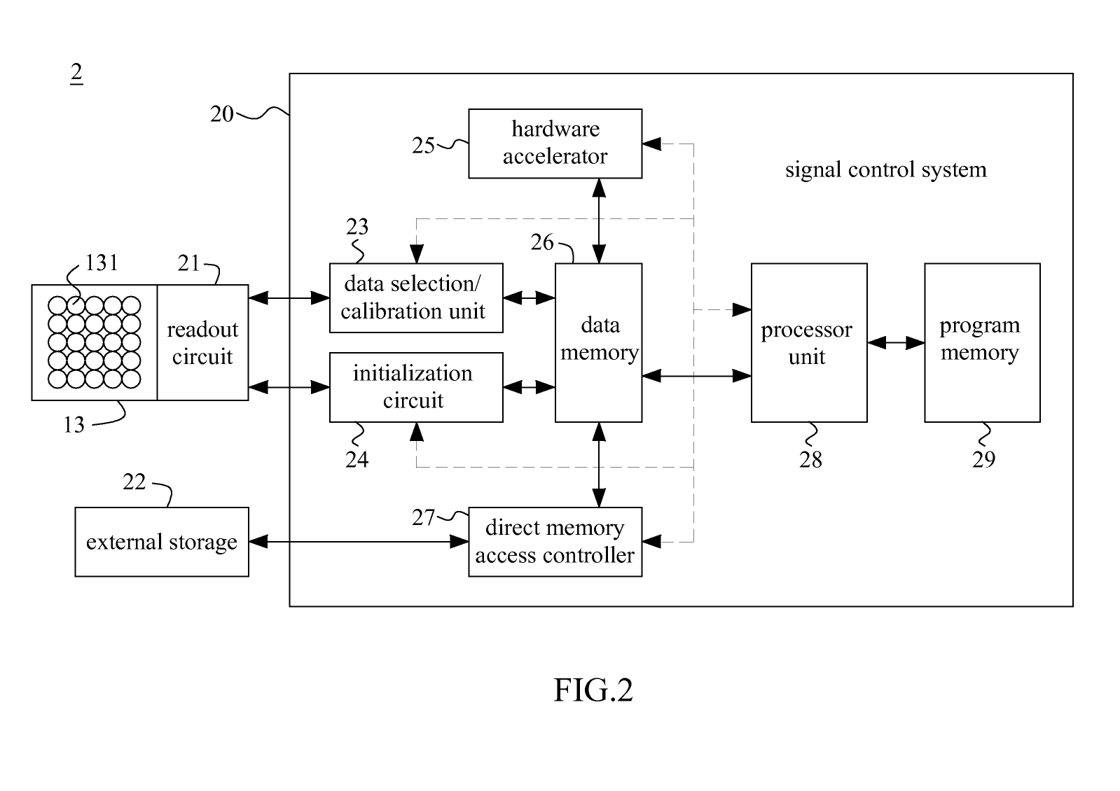

With the advances in the semiconductor process technology, the concept of system-on-a-chip (SoC) which integrates all circuit functional blocks in a single chip has been realized, becoming the trend for the development of electronics systems. The hemodynamic monitoring device 1 has been proposed for non-invasive carotid blood flow test based on the SoC approach. FIG. 2 is a system architecture diagram illustrating a non-invasive array-based hemodynamic monitoring SoC according to one embodiment of the present invention. As shown in FIG. 2, the non-invasive array-based hemodynamic monitoring SoC 2 comprises a CMOS MEMS pressure sensor array 13, a readout circuit 21, and a signal control system 20.

The two-dimensional CMOS MEMS pressure sensor array 13 comprises a plurality of CMOS compatible MEMS pressure sensors 131 which are configured to sense the pulse wave of the blood vessel. The CMOS compatible. MEMS pressure sensors 131 are micro-electro-mechanical systems (MEMS)-based pressure sensors, which are compatible with start-of-the-art CMOS process. The process technology and design of the CMOS compatible MEMS pressure sensors 131 may be discussed in more detail below. The readout circuit 21 is coupled with each CMOS compatible MEMS pressure sensors 131 and is configured to read the pulse wave sensed by the CMOS MEMS pressure sensor array 13. The readout circuit 21 is the interface circuits to transform either capacitance change (ΔC) or resistance changes (ΔR), generated by the pressure sensors 131, into a voltage signal.

The signal control system 20 coupled with the readout circuit 21 comprises a data selection/calibration unit 23, initialization circuit 24, hardware accelerator 25, direct memory access controller 27, data memory 26, program memory 29, and processor unit 28. This processor unit 28, such as a digital signal processor (DSP), executes a waveform estimation algorithm that is designed to capture and estimate the waveform (like blood pulse waveform) of a high-velocity signal both temporally and spatially from the temporal waveforms sensed by the pressure sensors 131 on the sensor array 13 that is separated from the signal source with some known substance in between.

To estimate the waveform, the waveform estimation algorithm does the following steps:

1. Each CMOS compatible MEMS pressure sensors 131 of the CMOS MEMS pressure sensor array 13 detects the signal waveform temporally by sampling continuously.

2. From the temporal waveforms sensed by the pressure sensors 131, only keep the ones with the largest signal power. From the location of the associated sensor, we can detect the direction of the signal propagation (e.g., the direction of the blood vessel).

3. From the temporal waveform sᵢ(t) detected by the i-th pressure sensors 131 remaining in Step 2, estimate the signal velocity vector ν=(νₓ, ν_{y}) using our waveform velocity estimation schemes.

4. With the velocity ν of the signal waveform estimated, the spatial feature of the waveform can also be captured using the temporal information sᵢ(t) and the velocity vector of the waveform, by the expression (1).

sᵢ(t−[(x−xᵢ)²+(y−yᵢ)²]^{0.5}/[νₓ²+ν_{y}²]^{0.5}) (1)

where si(t) is the signal detected by the i-th pressure sensors 131 located at location (xi, yi).

In this waveform estimation algorithm, one needs to estimate the velocity of the waveform. There are three different schemes that can achieve the task.

Scheme I: Time Correlating Scheme

1. From the sensors that are aligned to the direction of signal propagation (obtained from Step 2 of our waveform estimation algorithm above), choose any two distinct sensors. Denote them as the p-th sensor 131 (located at (xₚ, yₚ)) and the q-th sensor 131 (located at (x_{q}, y_{q})).

2. From sₚ(t) and s_{q}(t), find the time difference n*Ts that causes the highest N-tap cross correlation of sₚ(t−n*Ts) and s_{q}(t), where Ts denote the sampling period of each sensor in sensing temporal waveform, i.e. the expression (2).

$${n}^{*} = {argmax}_{n} {\sum }_{j = 0}^{N} {s}_{p} ( ( j - n ) {T}_{s} ) \cdot {s}_{q} ( {jT}_{s} ) \qquad ( 2 )$$

3. The velocity can be estimated as v=(vₓ, v_{y}), where vₓ=(xp−xq)/n*/Ts, v_{y}=(yₚ−y_{q})/n*/Ts.

Scheme II: Spatial Correlating Scheme

1. From the sensors 131 that are aligned to the direction of signal propagation (obtained from Step 2 of the waveform estimation algorithm), choose the sensor pair p* and q* that has the highest N-tap cross correlation, i.e. the expression (3).

$$( {p}^{*} , {q}^{*} ) = {argmax}_{( p , q )} {\sum }_{j = 0}^{N} {s}_{p} ( ( j - 1 ) {T}_{s} ) \cdot {s}_{q} ( {jT}_{s} ) \qquad ( 3 )$$

2. From the coordinate of the p*-th sensor 131 (located at (xₚ*, yₚ*)) and the q*-th sensor 131 (located at (x_{q}*, y_{q}*)), the velocity can be estimated as ν=(νₓ, ν_{y}), where νₓ=(xₚ*−x_{q}*)/Ts, ν_{y}=(yₚ*−yₚ*)/Ts.

Scheme III: Bi-Directional Spatial Correlating Scheme

1. From the sensor array 13, choose two sensors 131 that are aligned to the x axis (horizontal direction) of the sensor array. Denote them as the p-th sensor 131 (located at (xₚ, yₚ)) and the q-th sensor 131 (located at (x_{q}, y_{q})). Note that yₚ=y_{q}.

2. From sₚ(t) and s_{q}(t), find the time difference n*Ts that causes the highest N-tap cross correlation of sₚ(t−n*Ts) and s_{q}(t), where Ts denote the sampling period of each sensor in sensing temporal waveform, i.e. the expression (4).

$${n}^{*} = {argmax}_{n} {\sum }_{j = 0}^{N} {s}_{p} ( ( j - n ) {T}_{s} ) \cdot {s}_{q} ( {jT}_{s} ) \qquad ( 4 )$$

3. The x component of the velocity can be estimated as νₓ=(xₚ−x_{q})/n*/Ts.

4. From the sensor array 13, choose two sensors 131 that are aligned to the y axis (horizontal direction) of the sensor array. Denote them as the p′-th sensor 131 (located at (xₚ′, yₚ′)) and the q′-th sensor 131 (located at (x_{q}′, y_{q}′)). Note that xₚ′=x_{q}′.

5. From sₚ′(t) and s_{q}′(t), find the time difference n*Ts that causes the highest N-tap cross correlation of sₚ′(t−n*Ts) and sq′(t), i.e. the expression (5).

$${n}^{′} = {argm ax}_{n} {\sum }_{j = 0}^{N} {s}_{p} ( ( j - n ) {T}_{s} ) \cdot {s}_{q} ( {jT}_{s} ) \qquad ( 5 )$$

6. The y component of the velocity can be estimated as v_{y}=(yₚ′−y_{q}′)/n′/Ts.

7. The velocity can be estimated as v=(vₓ, v_{y}).

Therefore, the wave velocity of the pulsatile pressure propagating in an artery can be estimated by executing the waveform estimation algorithm. Once the spatial and temporal pattern of the pulsatile pressure is identified, some healthy information such as the rate of blood flow in the artery, the temporal profile of the distension of the arterial wall, and the mechanical properties of the artery based on the spatial and temporal profile of pressure data can be derived. In order to derive the healthy information, the present invention provides a mechanical model representation of an inhomogeneous, viscoelastic medium encompassing a long, viscoelastic pipe, while the pipe is pressurized by a pulsatile flow from one end and partial pressure wave reflection occurs at the other end. The surface of the medium is away from the pipe at least twice of the size scale of the pipe's diameter and stress dissipation is allowed in the medium. A layer of material of the mechanical properties of the sensor array 13 is added at the boundaries of medium to simulate the presence of the sensor 131, but the contour, mechanical behavior of the pipe is not affected by the sensor-mimicking material. Finally, a transfer function is used to obtain the healthy information. In one embodiment, the transfer function may be, but is not limited to, the constitutive equations expressed as a Fourier series or a mapping table. The constitutive equations are developed based on fluid and solid mechanics to relate the mechanical response of the sensor 131 to the pulsatile pressure propagating in the artery. The system also can look up the pre-determined mapping table to get the corresponding healthy information of a specific given wave pattern of the pulsatile pressure.

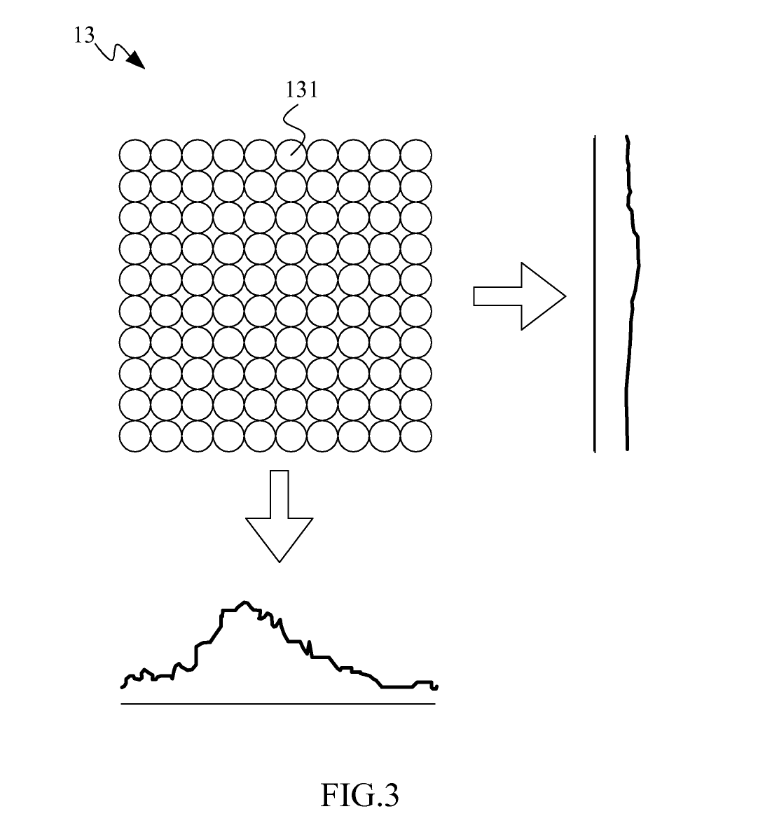

In order to realize a low-power medical system to monitoring for long-period, the data selection/calibration unit 23, initialization circuit 24, hardware accelerator 25, direct memory access controller 27, on-chip data memory 26, and program memory 29 are used. The initialization circuit 24 takes charge of detection basic parameters of the hemodynamic monitoring system. FIG. 3 is a diagram illustrating waveforms sensed by the sensors on a sensor array according to one embodiment of the present invention. As shown in FIG. 3, the signal change of waveform sensed by the sensor array 13 may be reflected from the position or direction of the blood vessel. The initialization circuit 24 can detect basic parameters with projection process to find the possible position of the blood vessel. It can determine the position of significant sensors 131, rather than process the signals of all sensors 131 in the sensor array 13.

The data selection/calibration unit 23 can use subsampling and undersampling techniques to select a particular region-of-interest waveform signal sensed by the sensors 131. The techniques can reduce the amount of data to be analyzed and achieve low-power mechanism. The data selection/calibration unit also can be dynamically adjusted the input signal with a look-up-table generated by a calibration sub-system. The data selection/calibration unit 23 is configured to sample waveform signal sensed by the sensors 131 on the CMOS MEMS pressure sensor array 13 with fixed or dynamically adjusted sampling period or interval, or both. Moreover, dedicated hardware accelerators 25, such as FFT/IFFT, are also integrated to accelerate some key operations with better power efficiency.

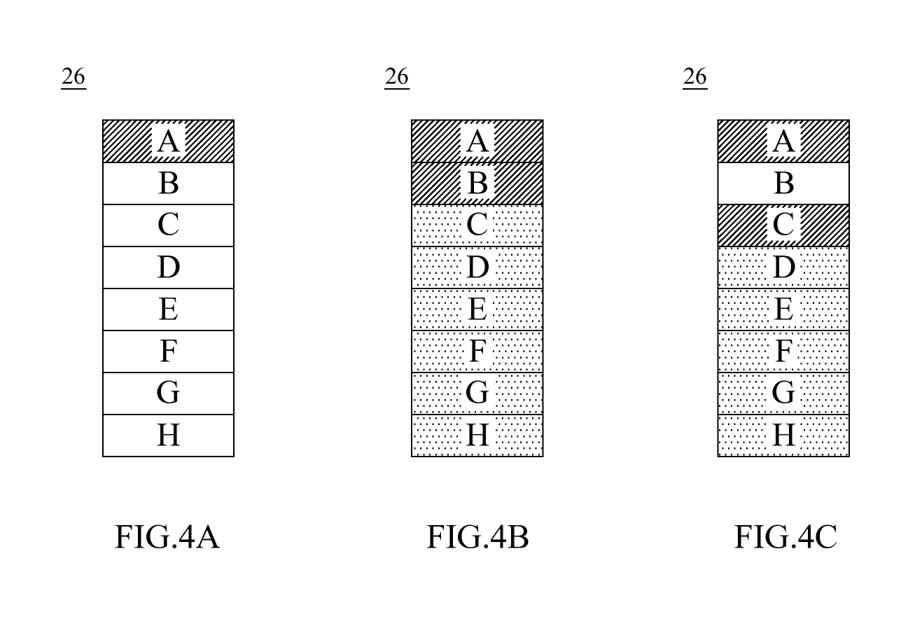

The on-chip data memory 26 is designed with multi-bank multi-operation-mode memory, which is optimized for this application in power consumption. Please refer to FIGS. 4A-4C, which show usage of multi-bank memory according to one embodiment of the present invention. The storage space of the data memory 26 is divided into a plurality of banks A-H. Three operation modes are pre-defined, that is, normal (active) mode, power-off (inactive) mode and low-power (idle) mode. The initial multi-bank memory is empty except the bank A, as shown in FIG. 4A. The bank A acts as general-purpose temporary registers, so it is always in the normal mode. The input data such as waveform signal sensed by the sensor array 13 are started to store from bank B to bank H. The first part of the input data in bank B will be analyzed, and the bank B enters into the normal mode, as shown in FIG. 4B. After the data in bank B is analyzed, it can be turned off to save power consumption and enters into the power-off mode. Later, the part of the input data in bank C will be analyzed, so the status of bank C is changed from the low-power mode to the normal mode, and so on.

The non-invasive array-based hemodynamic monitoring SoC 2 further comprises an external storage 22 such as a flash memory, which is used to store the waveform diagram sensed by the sensor array 13 or the analyzed result of the input data.

In one embodiment of the present invention, the signal control system 20 further comprises a pipelined-ADC calibration unit, which allows the user to set the number of available post-calibration ADC output codes. In a pipelined-ADC array in which the ADCs should have matched performance, this feature eliminates the post-calibration gain adjustment. The result is lower silicon overhead, less power consumption, and reduced latency.

The present invention uses a CMOS compatible process to develop MEMS-based pressure sensors 131 to provide an orthogonal sensing platform to further enhance the sensitivity, robustness, and accuracy of the pulse pressure measurement. Few post-processes will be designed and implemented after a typical CMOS MEMS process to complete our sensors' fabrication. In one embodiment, the manufacturing capability has been demonstrated by major CMOS foundries, e.g. TSMC or UMC. The mixed mode design concept with four basic pressure sensor designs with different sensing mechanisms comprises the capacitive based, piezo-resistive based, resonant-based, and piezo-electric based CMOS MEMS. After the circuits and MEMS-based sensors are partially completed with a typical process, focused ion beam (FIB) technology will be used to locally dope the silicon dielectric layer on the top to form a piezo-electric layer or piezo-resistive layer. In addition, the FIB technology can also be used to locally deposit inorganic material (such as metal) layer on the silicon dielectric layer for electrical interconnection. The mixed mode sensors can be done in several ways. In one embodiment, the resonant mode-based sensor can be combined with piezo-electric and/or piezo-resistive and/or capacitive-based sensing. The various embodiments can be designed based on the combination of different sensing mechanisms.

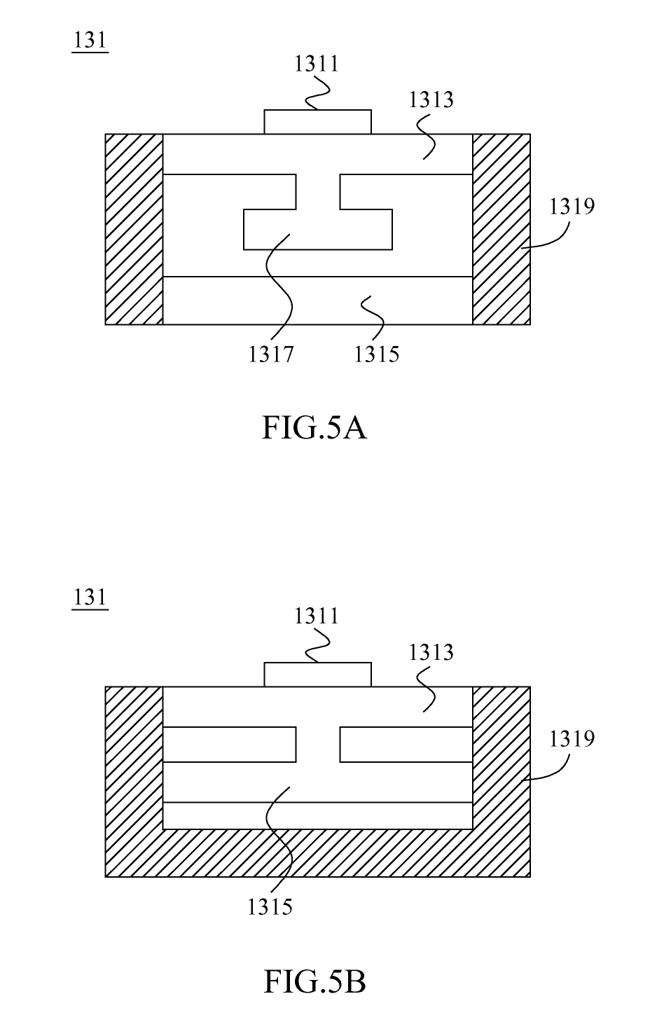

In one embodiment of the present invention, please refer to FIG. 5A, which shows a diagram illustrating design of a capacitive pressure sensor according to one embodiment of the present invention. As shown in FIG. 5A, the CMOS compatible MEMS pressure sensors 131 comprises a flange 1311, a top flexible plate 1313, a bottom electrode plate 1315, and a cushion electrode plate 1317. The flange 1311 is the point which touches the skin of the patient. When a pulse wave of a blood vessel reaches the flange 1311, the flange is pressed by the pulse pressure so as to press and bend the top flexible plate 1313. Then the distance between the bottom electrode plate 1315 and the cushion electrode plate 1317 changes thus the capacitance changes. Compared to other capacitive-based sensors with two parallel electrodes, our design is to have a uniform gap in between the bottom surface of the cushion electrode plate 1317 and the top surface of the bottom electrode plate 1315 when responding to an external pulse pressure. The pulse pressure difference is directly proportional to the gap variation in our device. In addition, when responding to the pressure differences, the precise value of the capacitance change can be measured with the uniform gap variation in between two sensing electrodes and thus an accurate output is obtained. The capacitance changing quantity between two plates 1315, 1317 will indicate the pressure quantity, which is uniform and easy to sense.

In another embodiment of the present invention, please refer to FIG. 5B, which shows a diagram illustrating design of a piezo-resistive pressure sensor according to one embodiment of the present invention. As shown in FIG. 5B, the CMOS compatible MEMS pressure sensors 131 comprises a flange 1311, a top flexible plate 1313, and a bottom electrode plate 1315. The flange 1311 is the point which touches the skin of the patient. When the pulse pressure applies to the flange 1311, the support beam will suffer an axial stress, thus change the resistance value of the strain gauge.

Based on the above, the CMOS compatible MEMS pressure sensor could detect many kinds of electrical characteristics from the top flexible plate, the bottom electrode plate, and the cushion electrode plate so as to determine the pressure quantity. The electrical characteristics could further comprise the resonant frequency changing quantity other than the foregoing capacitance and resistance changing quantities.

When MEMS combines with CMOS circuits, the manufacturing cost can be reduced. The development cost is reduced. This high resolution pulse pressure measurement will be calibrated with human hemodynamic modeling to obtain the blood flow measurement of main arteries inside the human body.

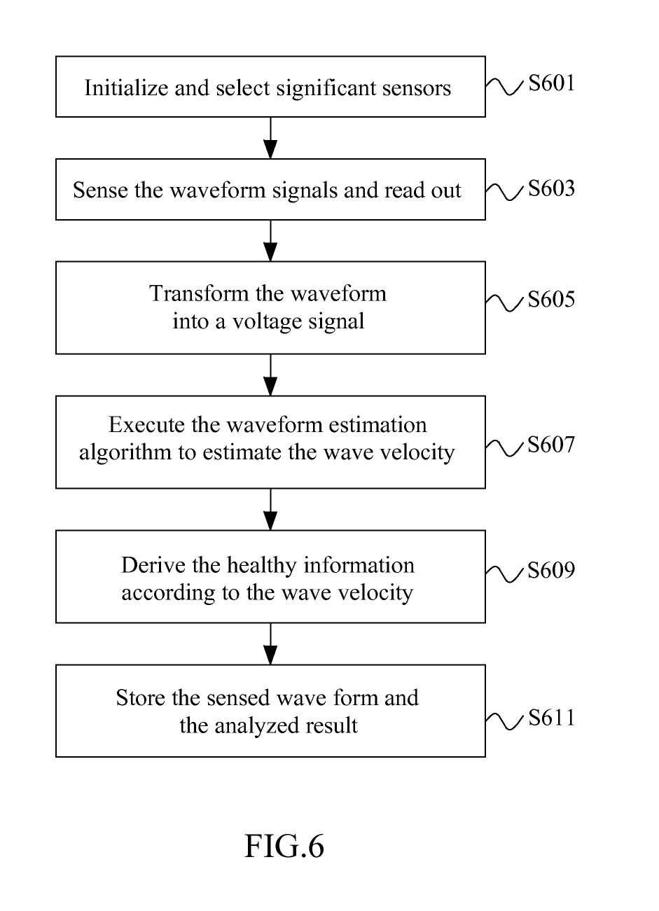

FIG. 6 is a flow diagram illustrating a non-invasive array-based hemodynamic monitoring method according to one embodiment of the present invention. The method comprises the following steps.

When first-time use of this setup or every time a new display device is used, the non-invasive array-based hemodynamic monitoring SoC 2 needs to be initialized with a calibration phase to select significant sensors 131 or do above sampling process. Firstly, in step S601, it initializes to find the possible position of the blood vessel and select significant sensors 131. Then, in step S603, the selected sensors 131 of the CMOS MEMS pressure sensor array 13 sense the waveform signals of the pulse wave, and the readout circuit 21 transforms the waveform signals into a voltage signal in step S605.

After receiving the voltage signal, the processor unit 28 executes the waveform estimation algorithm to estimate the wave velocity according to the voltage signal in step S607. Then, the processor unit 28 also derives the healthy information according to the wave velocity in step S609. Finally, the processor unit 28 may store the sensed wave form and the analyzed result such as wave velocity and healthy information in the external storage 22 in step S611.

Although specific embodiments have been illustrated and described, it will be appreciated by those skilled in the art that various modifications may be made without departing from the scope of the present invention, which is intended to be limited solely by the appended claims.

What is claimed is:

1. A non-invasive array-based hemodynamic monitoring system on chip, comprising:
a CMOS MEMS pressure sensor array configured to sense a pulse wave of a blood vessel;
a readout circuit, coupled with each of the CMOS compatible MEMS pressure sensors, configured to read the pulse wave and transformed the pulse wave into a voltage signal; and
a signal control system, coupled with each of the readout circuit, configured to estimate a wave velocity according to the voltage signal.

2. The system on chip of claim 1, wherein the signal control system comprises:
a processor unit configured to execute a waveform estimation algorithm to estimate the wave velocity;
wherein, the waveform estimation algorithm is designed to capture and estimate the waveform signal according to either temporally or spatially correlations, or both, from the temporal waveforms sensed by the CMOS MEMS pressure sensor array.

3. The system on chip of claim 2, further comprises:
a mechanical model configured to derive at least one healthy information according to a wave pattern; and
an external storage configured to store the sensed pulse wave, the wave velocity and the healthy information.

4. The system on chip of claim 3, wherein the mechanical model uses a transfer function to obtain the healthy information, the transfer function comprises the constitutive equations such as a Fourier series or a mapping table.

5. The system on chip of claim 3, wherein the signal control system further comprises:
a data memory divided into a plurality of banks to store the input data sensed by the CMOS MEMS pressure sensor array;
wherein, the bank enters into a normal (active) mode if the stored input data within is analyzed, the bank enters into a power-off (inactive) mode if the stored input data within has been analyzed, and the bank enters into a low-power (idle) mode if the stored input data within is waiting to be analyzed.

6. The system on chip of claim 1, wherein the signal control system further comprises:
an initialization circuit configured to find the possible position of the blood vessel and determine the position of significant sensors on the CMOS MEMS pressure sensor array; and
a data selection/calibration unit configured to select a particular region-of-interest waveform signal sensed by the sensors on the CMOS MEMS pressure sensor array.

7. The system on chip of claim 6, wherein the data selection/calibration unit is configured to sample waveform signal sensed by the sensors on the CMOS MEMS pressure sensor array with fixed or dynamically adjusted sampling period or interval, or both.

8. The system on chip of claim 1, wherein the CMOS MEMS pressure sensor array comprises a plurality of CMOS compatible MEMS pressure sensors.

9. The system on chip of claim 8, wherein the CMOS compatible MEMS pressure sensors comprises the capacitive based mechanism, piezo-resistive based mechanism, resonant-based mechanism, and piezo-electric based CMOS MEMS mechanism.

10. The system on chip of claim 8, wherein the circuits and MEMS-based sensors are partially completed with a typical process, focused ion beam (FIB) technology will be used to locally dope the silicon dielectric layer on the top to form a piezo-electric layer or piezo-resistive layer.

11. The system on chip of claim 8, wherein each of the CMOS compatible MEMS pressure sensors comprises:
a flange disposed to be touch;
a top flexible plate coupled to the flange;
a bottom electrode plate which is parallel with the top flexible plate; and
a cushion electrode plate coupled with the top flexible plate, and keeping flat between the top flexible plate and the bottom electrode plate;
wherein when the top flexible plate is pressed, the electrical characteristics from the top flexible plate, the bottom electrode plate and the cushion electrode plate indicates the pressure quantity.

12. The system on chip of claim 3, wherein the healthy information comprises the rate of blood flow in an artery, the temporal profile of the distension of the arterial wall, and the mechanical properties of the artery based on the spatial and temporal profile of pressure data.

13. A non-invasive array-based hemodynamic monitoring method, comprising:
providing a CMOS MEMS pressure sensor array to sense a pulse wave of a blood vessel;
transforming the pulse wave into a voltage signal; and
executing a waveform estimation algorithm to estimate a wave velocity according to the voltage signal.

14. The method of claim 13, wherein after the step of estimating the wave velocity comprises:
providing a mechanical model; and
deriving at least one healthy information according to the wave velocity.

15. The method of claim 14, wherein the step of deriving the healthy information comprises:
providing a mechanical model; and
using a transfer function to obtain the healthy information by the mechanical model;
wherein, the transfer function comprises the constitutive equations expressed as a Fourier series or a mapping table.

16. The method of claim 14, wherein the CMOS MEMS pressure sensor array comprises a plurality of CMOS compatible MEMS pressure sensors, and the method further comprises:
initializing to find the possible position of the blood vessel and select significant sensors from the CMOS compatible MEMS pressure sensors; and
sensing and reading out the wave form signals of the selected significant sensors.

17. The method of claim 16, further comprising:
storing the sensed wave form, the voltage signal, the wave velocity and the healthy information.

18. The method of claim 16, wherein the CMOS compatible MEMS pressure sensors comprises the capacitive based mechanism, piezo-resistive based mechanism, resonant-based mechanism, and piezo-electric based CMOS MEMS mechanism.

19. A CMOS compatible MEMS pressure sensor, comprising:
a top flexible plate;
a bottom electrode plate; and
a cushion electrode plate coupled with the top flexible plate and keeping flat between the top flexible plate and the bottom electrode plate;
wherein when the top flexible plate is pressed, the electrical characteristics from the top flexible plate, the bottom electrode plate and the cushion electrode plate indicates the pressure quantity.

20. The sensor of claim 19, wherein plural CMOS compatible MEMS pressure sensors are arranged to a two-dimensional CMOS MEMS pressure sensor array, which is used to sense a pulse wave of a blood vessel.

21. The sensor of claim 20, wherein the two-dimensional CMOS MEMS pressure sensor array is adapted to a non-invasive array-based hemodynamic monitoring system on chip, wherein the non-invasive array-based hemodynamic monitoring system on chip comprises:
a readout circuit, coupled with each of the CMOS compatible MEMS pressure sensors, configured to read the pulse wave and transformed the pulse wave into a voltage signal; and
a signal control system, coupled with each of the readout circuit, configured to estimate a wave velocity according to the voltage signal.

22. The sensor of claim 19, wherein the electrical characteristics comprise the capacitance changing quantity, the resistance changing quantity or the resonant frequency changing quantity.

23. The sensor of claim 22, wherein when the top flexible plate is pressed, the top flexible plate is bent to change the distance between the bottom electrode plate and the cushion electrode plate so as to result the capacitance changing quantity.

24. The sensor of claim 23, further comprising a flange disposed on the top flexible plate to be touch and the flange is pressed by a pulse wave of a blood vessel to bend the top flexible plate.

25. The sensor of claim 19, which the cushion electrode plate is parallel with the bottom electrode plate and is shorter than the top flexible plate.
